(19) United States
(12) Patent Application Publication
(10) Pub. No.: US 2010/0129912 A1
(43) Pub. Date: May 27, 2010

(54) 3D Cell-Culture Article and Methods Thereof

(76) Inventors: Hui Su, Big Flats, NY (US); Oleksandr Sydorenko, Cherry Hill, NJ (US)

Correspondence Address: CORNING INCORPORATED, SP-TI-3-1, CORNING, NY, 14831, US

(21) Appl. No.: 12/624,948

(22) Filed: Nov. 24, 2009

Related U.S. Application Data
(60) Provisional application No. 61/117,366, filed on Nov. 24, 2008.
(60) Provisional application No. 61/260,456, filed on Nov. 12, 2009.

(51) Int. Cl. C12N 5/02

(52) U.S. Cl. 435/402; 435/395

(57) ABSTRACT

An optically clear, porous polymer composition, an article incorporating the composition, and methods for making and using the composition for cell culture including, for example, regulating or promoting cell function or gene expression as defined herein.

### CLAIMING BENEFIT OF PRIOR FILED U.S. APPLICATION

This application claims the benefit of U.S. Provisional Application Ser. No. 61/117366, filed on Nov. 24, 2008 and U.S. Provisional Application Ser. No. 61/260456, filed on Nov. 12, 2009. The contents of this priority document and the entire disclosure of any publication, patent, or patent document mentioned herein is incorporated by reference.

### BACKGROUND

The disclosure is related to a cell-culture article and to methods for making the article and using the article in cell culture.

### SUMMARY

The disclosure provides a highly porous three dimensional (3D) composition having an interconnected network of pores and interstices, and articles incorporating the compositions, such as a cell-culture article having high optical clarity, such as when coated on a substrate, with or without culture media or cells present. The disclosure also provides a method of making the highly porous 3D composition and articles thereof, and methods for cell culture, including for example regulating cell function or gene expression, and cell culture monitoring with the articles.

### BRIEF DESCRIPTION OF THE DRAWINGS

In embodiments of the disclosure:

FIG. 1 shows an exemplary porous polydimethoxysilane (PDMS) article having an interconnected pore and interstice structure as imaged by a confocal microscope in reflection mode;

FIG. 2A shows a microscope image of a porous PDMS in surface plane focus;

FIG. 2B shows HepG2 spheroids that formed during culture within the porous PDMS cell culture article of FIG. 2A;

FIG. 3A shows an exemplary sample of a porous PDMS article having an interconnected pore structure imaged by a confocal microscope in reflection mode;

FIG. 3B shows a two-photon fluorescence image of the sample of FIG. 3A doped with Qdot at depth of 530 microns;

FIG. 4 shows an exemplary schematic representation of close-packed mono-modal large pore-former ensemble;

FIG. 5 shows an exemplary schematic representation of close-packed bi-modal large and small pore-former ensemble;

FIG. 6 shows cell attachment LDH assay evaluation results after 24 hours culture of hepatocyte cell line HepG2 C3A;

FIG. 7 shows the results of a cell attachment LDH assay after 7-day culture of HepG2 in comparative articles and in inventive porous article (PDMS);

FIG. 8 shows the results of an LDH assay of human primary hepatocytes cultures of CYP3A4 and CYP1A2 primary cells in a comparative collagen article and an inventive porous article (PDMS) with and without inducers;

FIG. 9 shows gene expression analysis results of human primary hepatocyte of the inventive composition against collagen as a normalization standard;

FIG. 10 compares the number of viable human hepatocyte cells after culture with various culture surfaces;

FIGS. 11A and 11B show gene expression levels of human hepatocytes induced by rifampin in porous PDMS substrates having various pore sizes.

FIG. 12 shows selective gene expression quality of human hepatocytes cultured in porous PDMS substrates having various pore sizes and having various stiffness (mix ratio).

FIG. 13 a shows a relationship of the measured modulus (stress versus strain) for various disclosed porous PDMS substrates.

FIG. 14 shows a relationship of the measured modulus of curves for the porous PDMS substrates in FIG. 13 as a function of the substrate stiffness as determined by the mixing ratio of the monomer or the oligomer material to curing agent.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, examples in this specification are not limiting and merely set forth some of the many possible embodiments for the claimed invention.

### Definitions

“Pore” refers to, for example, a cavity or void in the surface, the body, or both surface and body of the solid polymer having at least one outer opening at a surface of the polymer article.

“Interstice” refers to, for example, a cavity or void in the body of the solid polymer not having a direct outer opening at a surface of the polymer article, i.e., not a pore, but may have an indirect outer opening or pathway to an outer surface of the polymer object by way of one or more links or connections to adjacent or neighbor “pores”, “interstices,” or a combination thereof.

“Porous network” refers to, for example, the combined or total void-volume, consisting of the pores and the interstices, of the article remaining after the particulate material has been removed from the composition during manufacture in accordance with the disclosure.

“Porosity” refers to, for example, the ratio of the total interstitial volume of pores and interstices of a material to the volume of the material's mass.

“Continuous void phase” refers to an article having an interconnected porous network that is substantially free of “dead ends” or “no-outlets” such as having only a single connection to another interstice, or “isolated voids,” that is, interstices having no interconnectivity. A semi-continuous void phase refers to an article having an interconnected porous network that may have some amount of the above mentioned “dead ends” or “isolated voids,” such as from about 1 to about 20% by volume. When a semi-continuous void phase having the abovementioned network properties is encountered or desired, pore-former selection can consider, for example, fugitive pore-formers such as a diffusible gas or a sublimable solid, or a hollow and transparent particle that is optically matched or similar to the polymer phase.

“Reconstitutable powder” as disclosed herein refers to a powder which when treated with a liquid produces an aggregated polymer mass having the disclosed porous interconnected network properties.

“Optical density,” “OD,” or like terms refers, for example, to a measure of the transmittance of porous polymer material of the inventive article for a given length at a given wavelength, in the presence or absence of culture media.

“Retention rate” refers to the percentage of the viable cell numbers plated to the culture surface that attach or stay on the surface after a period of time.

“Inducer” refers to a molecule or like agent that can cause a cell or an organism to accelerate biosynthesis of an enzyme or sequence of enzymes in response to a developmental signal.

“Assay,” “assaying” or like terms refers to an analysis to determine, for example, the presence, absence, quantity, extent, kinetics, dynamics, or type of a cell's growth characteristics or response to an exogenous stimuli, such as a ligand candidate compound, culture media, substrate coating, or like considerations.

“Attach,” “attachment,” “adhere,” “adhered,” “adherent,” “immobilized”, or like terms generally refer to immobilizing or fixing, for example, a surface modifier substance, a compatibilizer, an inducer, a cell, a ligand candidate compound, and like entities of the disclosure, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with cells with a surface, such as a biosensor surface (such as a Corning, Inc., Epic® instrument or like devices) or a culture surface.

“Adherent cells” refers to a cell or a cell line or a cell system, such as a prokaryotic or eukaryotic cell, that remains associated with, immobilized on, or in certain contact with the outer surface of a substrate. Such type of cells after culturing can withstand or survive washing and medium exchanging process, a process that is prerequisite to many cell-based assays. “Weakly adherent cells” refers to a cell or a cell line or a cell system, such as a prokaryotic or eukaryotic cell, which weakly interacts, or associates or contacts with the surface of a substrate during cell culture. However, these types of cells, for example, human embryonic kidney (HEK) cells, tend to dissociate easily from the surface of a substrate by physically disturbing approaches such as washing or medium exchange. “Suspension cells” refers to a cell or a cell line that is preferably cultured in a medium wherein the cells do not attach or adhere to the surface of a substrate during the culture. “Cell culture” or “cell culturing” refers to the process by which either prokaryotic or eukaryotic cells are grown under controlled conditions. “Cell culture” can include the culturing of cells derived from multicellular eukaryotes, especially animal cells, but also to the culturing of complex tissues, organs, pathogens, or like systems.

“Cell” or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

“Cell system” or like term refers to a collection of cells and can include more than one type of cells (or differentiated forms of a single type of cell), which interact with each other, thus performing a biological, physiological, or pathophysiological function. Such cell system can include, for example, an organ, a tissue, a stem cell, a differentiated hepatocyte cell, and like cell systems.

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, and like abbreviations).

“Weight percent,” “wt. %,” “percent by weight,” or like terms with reference to, for example, a component, unless specifically stated to the contrary, refer to the ratio of the weight of the component to the total weight of the composition in which the component is included, expressed as a percentage.

“Include,” “includes,” or like terms means comprising or including but not limited to, that is inclusive and not exclusive.

“About” modifying, for example, the quantity of an ingredient in a composition, concentrations, volumes, process temperature, process time, yields, flow rates, pressures, and like values, and ranges thereof, employed in describing embodiments of the disclosure, refers to, for example, variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities.

“Consisting essentially of” in embodiments refers, for example, to a composition, a method of making or using a composition, formulation, or a composition on the surface of a substrate, a cell culture article, and like articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular components, particular additives or ingredients, a particular agents, a particular cell or cell line, a particular surface modifier or condition, a particular ligand or drug candidate, or like structure, material, or process variable selected. Items that may materially affect the basic properties of the components or steps of the disclosure or that may impart undesirable characteristics to the present disclosure include, for example, an optical mismatch between the cell culture composition or article and the liquid culture media, optically mismatched or opaque entrapped pore-former particles which cannot be substantially removed from the composition, pore-former particles which can biologically, chemically, or optically contaminate the cell culture composition or article, and like considerations and characteristics.

The indefinite article “a” or “an” and its corresponding definite article “the” as used herein means at least one, or one or more, unless specified otherwise.

“Optional,” “optionally,” or like terms refer to the subsequently described event or circumstance can or cannot occur, and that the description includes instances where the event or circumstance occurs and instances where it does not. For example, the phrase “optional component” means that the component can or can not be present and that the disclosure includes both embodiments including and excluding the component.

Specific and preferred values disclosed for components, ingredients, additives, cell types, pathogens, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

The disclosure provides a non-animal based cell culture composition, article, such as for use with mammalian cells, and like cells, having cell function that more closely resembles in vivo-like behavior. For example, the in vitro culture of hepatocytes can be useful in the drug discovery processes (e.g., predictive ADMETox) since drugs can be converted to more toxic intermediates and interact with other compounds (e.g., drugs) after metabolism by the Cytochrome (CY)P450 enzyme in the liver as part of the detoxification process. However, primary hepatocyte cells in vitro can quickly lose function, including albumin production and CYP450 activity, which largely controls the cells ability to metabolize drug molecules. Heretofore, toxicity and drug interaction studies with these cells were typically limited and less informative.

In embodiments, the disclosure provides a synthetic composition for 3D cell culture, cell detection, or cell monitoring in 3D culture. This composition provides a cell culture environment for cells to grow and develop their natural functionality outside living entities, and these cell culture composition can be selected or designed to have suitable optical specifications which can enhance detection of cell activities in the substrates by, for example, having improved cell imaging penetration depth.

In embodiments, the porous polymer articles of the disclosure are useful for 3D cell culture and provide one or more of the following features, alone or in combination. Cultured cells can freely migrate, communicate, or contact one another through the interconnected porous structure of the article. Cultured cells can grow into spheroids of certain well defined sizes within an interstice or a pore in the porous article. The size of the spheroids can be controlled by defining the interstice or pore distribution of the substrates by judicious selection of the pore-former materials. In embodiments, porous articles having two (i.e., bimodal) or more (e.g., multi-modal) particle size distributions comprising the interstices of the porous network can be made to enhance the communication among cells and can enhance nutrient penetration into and waste transport out-of the interconnected channels of the network within the porous article. The porous articles having larger interstice and pore sizes can be designed to accommodate the cell or cell body growth, and the smaller interstice and pore sizes can be designed to enhance, for example, cell communication, nutrient exchange, and waste exchange. The porous articles are, for example, porous solids or porous gels, which can be easily combined-with and separated-from culture media including convenient continuous or semi-continuous culture media exchange. If desired, the porous articles can be made to be nearly transparent when immersed in culture media by matching the article's refractive index with or in close proximity to that of the media. A nearly transparent article enables, for example, deeper penetration for optical imaging of the cells residing inside the article. The imaging penetration of two-photon fluorescence microscopy in the inventive articles made of polydimethylsiloxane (PDMS) can reach, for example, from about 100 to about 1,000 microns, and deeper than about 500 microns compared to, for example, only about 90 microns in a polyvinylalcohol (PVA) based porous article. In embodiments, the disclosure provides an optical transparency solution even for polymers such as PVA having less desirable optical properties.

PDMS is a very stable and biologically compatible material for cell growth. Recently, Corning, Inc., introduced UltraWeb™ surfaces for 3D cell culture (see www.corning.com/Lifesciences/technical_information/techDocs/UltraWeb_References .pdf). UltraWeb™ is a synthetic membrane consisting of nano fiber structure. Cells spread and proliferate on top of the membrane surface. The present disclosure provides an improved 3D cell culture article and method having superior properties compared to an in vivo 3D cell culture system based on a cell culture system having UltraWeb™ surfaces.

The disclosure also relates to a porous cell culture article and methods for making and using the article. The cell culture article provides a three-dimensional environment (3D cell culture) having useful culture and optical detection properties of the cultured cells.

In a living entity, cells typically grow in three dimensions with a support structure of extra cellular matrix (ECM). The ECM contains, for example, proteins, such as collagen, elastin and laminin, which provide a mechanical support for cell growth in 3D as well as enable the communications between cells while growing and developing their functionalities. Cell biologists, especially cancer researchers have long suspected that the traditional monolayer or two dimensional (2D) cell culture on the flat surface of a petri dish is not sufficiently sophisticated to reproduce the growing environment necessary for cells to fully develop their natural biological activities and functionalities. Research interests in such 3D cell culture systems that can closely resemble the in-vivo cell growing structure began decades ago; however, the change of momentum for the 3D cell culture systems didn't come until Bissell's group demonstrated (see V. W. Weaver, et. al., Journal of Cell Biology, V137 (1), p231-245, 1997) the reversal of breast cancer cells cultured in a 3D culture system, which was never observed with the cells cultured in a 2D culture system. Since then, 3D cell culture systems quickly became an important alternative to traditional monolayer cell culture system.

Biomaterials derived from animal tissues, such as collagen gels (see H. K. Kleinman, et al., Biochemistry 21, p6188-6193, 1982) and Matrigel® (see Bell, E., Ivarsson, et. al., Proc. Natl. Acad. Sci.,76, p. 1274-1278, 1979), have been commonly used as ECM in 3D cell culture by researchers. Although these materials have proven effective for 3D cell culture and amenable to imaging cells down to a few hundred microns deep inside the matrix, extracts of animal tissues present the following disadvantages, including for example:
uncontrollable and undefined compositions of the substrates from batch-to-batch, which makes it difficult to compare the culture results of the substrates from different origins;
the sizes of cell spheroids formed in the matrix are random, which presents a difficulty to quantify results; and
the gel format of the matrix introduces difficulty in physical manipulation of the culture body, for example, changing media during the culture can be challenging without disturbing the cell culture body. Therefore, various synthetic materials based cell culture substrates, have been developed as an alternative to the natural substrates for 3D cell culture with limited success. Some illustrative examples include: a synthetic polymer consisting microfibers on a micro- or nano-scale demonstrated successful cell culture (see E. Entcheva, et al., Biomaterials, 25(26), P5753-5762, 2004; C. E. Semino, et al., Differentiation, 71, p262-270, 2003), but the depth of cell growth is limited and similar to two dimensional cell culture; porous polymer materials with pore size less than the cell diameter support the formation of a cell spheroid on the surface, but again they provide limited growth depth which is similar to the nano or micro fiber matrices; macro porous matrices made with synthetic material such as poly(lactic acid) (PLA) and poly(glycolic acid) (PGA) (see D. Barrera, et al., Copolymerization and degradation of poly(lactic-co-lysine), Macromolecules, 28, p425-432, 1995; G. Vunjak-Novakovic, et al., Dynamic cell seeding of polymer scaffolds for cartilage tissue engineering, Biotechnol. Prog., 14, p. 193-202, 1998), provide a structure for cells to grow into a greater depth than the fiber based substrates. However, these materials are opaque, and even the most advanced imaging techniques, such as confocal fluorescence microscopy and two-photon fluorescence, can image no deeper than a few hundred microns into the substrates. Thus, an ideal 3D cell culture system should provide a suitable three dimensional extra cellular matrix (ECM) or scaffold to support the cell growth in three dimensions, but should also enable the subsequent detection of cells in the culture matrix. The currently available 3D cell culture systems focus largely on providing a support for cell growth in 3D, giving little considerations to the subsequent detection of cells in the culture. The present disclosure provides a 3D cell culture system that permits cell growth or maintenance, and additionally provides for the convenient detection, study, or monitoring of cells in the culture.

The problem of limited cell culture viability and the problem of limited visibility in monitoring cell culture are solved by providing a highly porous cell-culture composition and article of the disclosure having a three dimensional (3D) interconnected porous network and having high optical clarity.

In embodiments the disclosure provides a method for making a three-dimensional porous cell culture article, the method comprising:
polymerizing a mixture comprising at least one monomer or oligomer, such as a dimethylsiloxane prepolymer or precursor and a siloxane prepolymer curing agent, cross-linker, or like catalyst, and at least one particulate pore-former, to form a continuous polymer matrix having a particulate phase; and
treating the resulting solid matrix to remove the particulate phase from the matrix.
The particulate phase in the polymer matrix can be, for example, a continuous phase, a semi-continuous phase, a discontinuous phase, or a combination thereof.

In embodiments, the at least one particulate pore-former can be, for example, at least one of:
a first particle mixture having a particle diameter of from about 75 micrometers to about 1,000 micrometers;
a second particle mixture having a particle diameter of from about 0.1 micrometers to about 75 micrometers; or
a combination thereof.

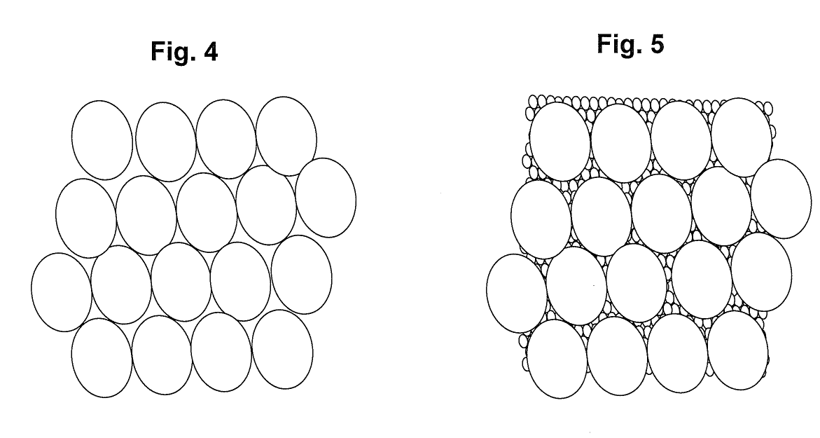

In embodiments, the pore-former can comprise, for example, a mixture of like or different particles having a mono-modal particle size distribution. Depending on the size distribution properties, the mono-modal distribution can provide a particle phase having large, intermediate, or small interstices, or a mixture thereof and corresponding large, intermediate, small surface pores, or a mixture thereof after removal of the particulate phase. FIG. 4 shows a schematic representation of close-packed mono-modal large pore-former ensemble.

In embodiments, the pore-former can comprise, for example, a mixture of particles having, for example, a bimodal particle size distribution. The bimodal particle size distribution having appropriate amounts of each mode can provide a particle phase having a mixture of large and small interstices, a mixture of large and small surface pores, or a mixture thereof. FIG. 5 shows a schematic representation of a close-packed bi-modal ensemble having a mixture of large and small pore-formers.

In embodiments, having a first particle mixture and the second particle mixture, the respective mixtures can be independently selected from, for example, mono-modal particles, bimodal particles, mono-disperse particles, bi-disperse particles, poly-disperse particles, and a combination thereof. In embodiments, the first particle mixture and the second particle mixture can be comprised of a same substance, or a different substance, yet having different particle size properties, particle size distribution properties, or a combination thereof. In general, as pore-former content increases, porosity and pore size increase, for example, in a linear fashion.

The polymerizing of the mixture can be accomplished on a suitable substrate. Additionally or alternatively, polymerizing the mixture can be accomplished as, for example, a pre-form, which is a molded form in a variety of useful shapes, and optionally attached to or associated with, for example, a substrate, a vessel, or like supports, i.e., polymerizing a mixture comprising at least one monomer or oligomer and at least one pore-former particulate material on a substrate to form a continuous polymer matrix and a discontinuous particulate phase on the substrate.

Polymerizing the at least one monomer or oligomer includes, for example, forming a continuous polymer phase of at least one monomer or oligomer selected from, for example, a siloxane, a vinyl substituted trialkoxy silane, an alpha-olefin, a vinyl ester, an acrylate, an acrylamide, an unsaturated ketone, a monovinylidene aromatic hydrocarbons, and like polymerizable monomer or oligomer, or a combination thereof. Examples of suitable monomer or oligomer which can be polymerized or copolymerized to form the articles as disclosed herein include the monovinylidene aromatic hydrocarbons (e.g., styrene, aralkylstyrene, such as the o-, m- and p-methylstyrenes, 2,4-dimethylstyrene, the Ar-ethylstyrenes, p-butylstyrene, and like monomer or oligomer; and alpha-alkylstyrene, such as alpha-methylstyrene, alpha-ethylstyrene, alpha-methyl-p-methylstyrene, and like monomer or oligomer; vinylnaphthalene, and like monomer or oligomer); Ar-halo-monovinylidene aromatic hydrocarbons (e.g., o-, m- and p-chlorostyrenes, 2,4-dibromostyrene, 2-methyl-4-chlorostyrene, and like monomer or oligomer); acrylonitrile, methacrylonitrile, alkyl acrylates (e.g., methylacrylate, butyl acrylate, ethylhexyl acrylate, and like monomer or oligomer), the corresponding alkyl methacrylates, acrylamides, (e.g., acrylamide, methylacrylamide, N-butylacrylamide, and like monomer or oligomer); unsaturated ketones (e.g., vinyl methyl ketone, methyl isopropenyl ketone, and like monomer or oligomer); alpha-olefins (e.g., ethylene, propylene, and like monomer or oligomer); vinyl esters (e.g., vinyl acetate, vinyl stearate, and like monomer or oligomer); vinyl and vinylidene halides (e.g., the vinyl and vinylidene chlorides, and bromides, and like monomer or oligomer); a vinyl substituted silane such as a vinyl substituted trialkoxy silane, and like monomer or oligomer, or combinations thereof. Hosoya, et al., in “High-Performance Polymer-Based Monolithic Capillary Column,” Anal. Chem., 2006, 78 (16), 5729-5735, mentions a capillary column was prepared using an epoxy monomer, Tris(2,3-epoxypropyl) isocyanurate (TEPIC), with diamines 4-[(4-Aminocyclohexyl)methyl]cyclohexylamine (BACM) and a chiral trans-1,2-cyclohexanediamine (CHD); and Tsujioka, et al., in “A New Preparation Method for Well-Controlled 3D Skeletal Epoxy Resin-Based Polymer Monoliths,” Macromolecules, 2005, 38 (24), 9901-9903, mentions that bis-phenol A digycidyl ether (BADE), (BACM), and a porogenic solvent such as poly(ethyleneglycol)(PEG) were used to prepare the 3D monoliths.

The at least one pore-former can be, for example, particles of a simple sugar, a polysaccharide, a polyalkylene glycol, a polyvinylalcohol, ice, a wax, a sublimable material such as solid CO₂, a substance having a melting point lower than that of the polymer formed, a water-soluble polymer, a water-insoluble polymer, or a copolymer thereof, a microcapsule having a shell and core where, for example, the shell comprises a monomer or oligomer insoluble material and the core comprises a water-miscible or water-soluble material, a micro-balloon having a soluble shell and hollow or gas filled core, or combinations thereof.

Treating of the resulting polymerized solid matrix to remove the particulate phase from the matrix can include, for example:
contacting with a substance to dissolve the particulate phase, the substance comprises at least one of: an aqueous liquid; an organic liquid; a supercritical fluid, e.g., CO₂; a low melting solid, e.g., wax, water, and like low melting solids; a gas, e.g., air, N₂, argon, and like gases; or a combination thereof;
heating the matrix to liquefy or dissolve the particulate phase;
or a combination of contacting and heating.
The resulting polymer phase less the particulate phase can have a refractive index for example, equal to or less than about 1.49, such as from about 1.2 to about 1.49 including all intermediate values and ranges, such as 1.2 to 1.4, 1.2 to 1.35, 1.25 to 1.4, 1.3 to 1.49, 1.3 to 1.4, 1.35 to 1.49, 1.35 to 1.4, and like values and ranges.

In embodiments, the preparative method can further comprise, for example, selecting a pore-former packing density based on a particle size ensemble having a void volume that becomes the continuous polymer matrix and the volume-fraction occupied by the particulate pore-former that becomes the void-volume, i.e., the combined interstice and pore-volume, in the resulting cell culture article.

The mixture comprising at least one monomer or oligomer for polymerization and at least one particulate pore-former can be prepared by, for example, at least one of: high speed liquid-solid mixing, liquid-solid blending, liquid-solid centrifugation, or a combination thereof.

In embodiments the disclosure provides a three-dimensional cell culture article including, for example:
a polymer mass having an interconnected porous network, the interconnected porous network comprising pores comprised of a single distribution of pore size or a bimodal distribution of larger and smaller sized pores and their corresponding interstices. In embodiments, the polymer mass of the article can be, for example, at least one of: a bead; a reconstitutable powder; a coating formulation, for example, a liquid suspension of particles of the porous polymer mass; a thin film of thickness of from 20 micrometers to 500 micrometers, a thick film of thickness of from 10,000 micrometers to 100,000 micrometers or more, or a film of intermediate thickness of thickness of from 500 micrometers to 10,000 micrometers, or combinations thereof.

In embodiments the disclosure provides a three-dimensional cell culture article prepared by the abovementioned process including:
polymerizing a mixture comprising at least one monomer or oligomer and at least one particulate pore-former to form a continuous polymer matrix having a distinct particulate phase; and
treating the resulting solid matrix to remove the particulate phase from the matrix.

The three-dimensional cell culture article can comprises, for example, a substrate; and a polymer layer having an interconnected porous network supported on the substrate, the porous polymer layer comprising a continuous polymer matrix having a continuous or semi-continuous void phase. In embodiments, the cell culture article can be characterized as being a bi-continuous material, i.e. the polymer forms a continuous matrix and the particulate voids form a second continuous, albeit hollow or open phase.

In embodiments, the porous polymer article can have a surface area of, for example, from about 0.1 to about 20 m²/g.

In embodiments, the porous polymer article can have a porosity of, for example, of from about 50% to about 95%, including intermediate values and ranges, as measured by mercury or nitrogen porosimetry; the porous polymer article can have, for example, a refractive index in air of, for example, from about 1.28 to about 1.49, including intermediate values and ranges; and the porous polymer article can have a density of, for example, from about 1 to about 1,000 kg/m³, including intermediate values and ranges.

In embodiments, the refractive index of a polydimethylsiloxane porous polymer can be, for example, from about 1.28 to about 1.49, the refractive index of a typical aqueous cell culture media can be, for example, from about 1.33 to about 1.36. In embodiments, the refractive index of porous polymer and the refractive index of a typical aqueous cell culture media can be selected so that there is a match or near match of the respective refractive indices, for example, where the difference in the respective refractive indices is less than about ±0.2 units, preferably less than about ±0.15, more preferably less than about ±0.12, and even more preferably less than about ±0.10. The porous polymer article can have an optical density of, for example, from about 0 to about 1, and an optical penetration depth of, for example, from about 100 to about 1,000 microns or more. The porous polymer article can comprise a polymer, copolymer, or like material, having a molecular weight of from about 500 to about 500,000 Daltons.

In embodiments, the article can further comprise at least one additive selected from the group consisting of a nutrient, an antibiotic, a growth stimulator, a growth inhibitor, a surface modifier, a surface compatibilizer, and like cell culture components such as one or more promoter, inhibitor, regulator, moderator, inducer, or a combination thereof.

In embodiments the disclosure provides a cell culture method including, for example: contacting the above mentioned cell culture with an article including a substrate, and a polymer layer having an interconnected porous network supported on the substrate, the porous polymer layer including a continuous polymer matrix having a continuous or semi-continuous void phase, and suitable culture media and culture conditions, such as temperature control, media exchange, and live-cells.

The cell culture provides a retention rate of from about 70 to about 100 percent compared to the retention rate of an industry standard collagen surface.

In embodiments, one example of a suitable cell line includes human primary cells. In embodiments, the culture article provides excellent cell line performance, cell functionality, cell viability, cell gene expression, and like properties, or a combination thereof.

To develop a 3D cell culture substrate that can assist cell culture and cell detection, it is desirable to match the optical properties of the base materials with the desired detection or imaging techniques used to subsequently monitor the cells in the culture. In embodiments of the disclosure, a fluorescence microscope can be used to monitor the cells in the cultures. Consideration should be given to the following optical properties of the base materials to construct the disclosed cell cultures: good optical transparency in the working wavelength range to avoid optical interference from the cultures during optical detection; and a refractive index that closely matches that of the cell culture media to decrease the optical scattering from the materials during detection. In addition to optical properties of the porous material for the article, the chemical properties of the porous materials can be selected to be very stable under culture conditions so that the cell growth will not be subject to changes of the article during the culture process. In embodiments, the porous material preferably has good gas and water permeability to facilitate the cell growth.

In embodiments, the base polymer material can be formed into a highly porous structure having interconnected pores or interstices through, for example, a forced filling and leaching process. In the forced filling process, fillers or pore-formers of desired sizes are homogenously mixed with the base polymer material, and either gravity or pressure can be used to force the fillers into close contact with each other and become closely packed with the monomer or oligomer(s), pre-polymer, or like precursor to the resulting polymerized or cured base polymer material. The resulting base polymer material packed with fillers can be treated to remove the fillers, such as by leaching-out or dissolving in an ultrasonic bath with an appropriate selective solvent. The pore size distribution of the porous substrate can be controlled by the sizes of the fillers, which can be one size distribution or more than one size distribution depending on the desired culture application. In addition to size consideration, the fillers used should also present minimum toxicity or interference to the cell culture physiological conditions so that any residual unleached fillers remaining in the porous substrates will not have a negative effect on cell growth.

Pore-former particles of a desired particle size and size distribution can be obtained by any suitable method including, for example, particle size reduction methods, particle size growth methods, or a combination thereof. A particle size reduction apparatus can be used for dry solid particles or particles suspended in a carrier liquid. One liquid-based particle-size reduction apparatus using high pressure liquid streams is, for example, a Microfluidizer®, or like apparatus, having an intensifier modification as mentioned in WO/2006/064203, entitled PARTICLE-SIZE REDUCTION APPARATUS, AND USE THEREOF, or like sizing apparatus, can be used to make desired particles by size reduction.

Particle size growth methods can include, for example, emulsion or suspension polymerization processes, or like particle size growth methods. The reduced-size particles or increased-size particles can be separated according to particle size or diameter ranges by, for example, a classifier, a filter, a screen, or like apparatus.

Desired particle size and size distribution can be measured and characterized by any suitable particle analyzer apparatus and method, such as available from Horiba (www.horiba.com), including for example, laser diffraction, dynamic light scattering, image analysis, or like sizing apparatus and method. The Horiba LB-550 can measure particle sizes from 1 nm to about 6 microns and at concentration ranges from ppm up to about 40% solids. The Horiba LA-300 Laser Diffraction

Particle Size Distribution Analyzer can be used to measure the particle size of suspensions or dry powders.

The culture article manufacture and procurement of accompanying components, such as a substrate and packaging materials, can preferably be accomplished under sterile conditions.

In embodiments, the present disclosure provides a non-animal sourced, cell culture coatings and its corresponding coated substrates that provides an in vivo-like cell culture.

In embodiments, the disclosed porous coatings and articles can be readily prepared and are relatively inexpensive. The disclosed coatings provide substrate coatings that are non-animal derived and the resulting coated products have, for example, little or no lot-to-lot variability, and excellent storage and shelf- or biological-stability. In embodiments, the disclosed porous polymer coatings can provide substrate surface coatings having a refractive index that can be easily tuned by, for example, selection of monomer or oligomer used in the polymer coating. The disclosed coating compositions can provide substrate surface coatings that can be non-toxic and biocompatible. The disclosed coating compositions can provide substrate surface coatings that are highly processible, such as easy to deposit onto a variety of surfaces, and provide enhanced adhesion of the coating and of cells to various substrates, for example, plastic, glass, and like cell culture substrates or supports. If desired, a tie-layer or conversion coating, such as an aminosilane can be selected to enhance adhesion of the porous polymer to a substrate such as glass.

In embodiments the disclosure provides a method for cell culture comprising:
providing a substrate coated with the abovementioned porous composition, or like compositions;
contacting the coated substrate with a cell culture for a sufficient time to establish functional cells;
monitoring the cells by optical methods; and
optionally harvesting the cells from the substrate.

In embodiments, the substrate can be, for example, a material selected from a metal oxide, a mixed metal oxide, a synthetic polymer, a natural polymer, like materials, or a combinations thereof. In embodiments, the porous cell culture articles of the disclosure can be further processed by, for example, surface treatment or conditioning to obtain articles having greater biocompatibility, see for example, commonly owned U.S. Pat. No. 6,617,152, and copending U.S. Ser. No. 11/973,832.

The cells of the cell culture can be, for example, any suitable primary cells, or associated immortalized cell lines, of any cell type such as hepatocytes. The cell culture can include, for example, cells actively producing albumin, antibodies, or like entities, and combinations thereof. The harvesting of cells from the substrate can be accomplished by any suitable means including, for example, centrifugation, agitation, washing, and like processes, or a combination thereof.

In embodiments, various biocompatible polymer materials can be selected for use in preparing the porous compositions. Additionally or alternatively, the biocompatible polymer materials can be used alone or in combination or in admixture with other cell culture materials or support materials. The polymer materials can include, for example, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, and copolymers thereof, nitro celluloses, polymers of acrylic and methacrylic esters, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, cellulose sulfate sodium salt, poly(methylmethacrylate), poly(ethylmethacrylate), poly(butylmethacrylate), poly(isobutylmethacrylate), poly(hexylmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate), poly vinyl chloride, polystyrene, polyhyaluronic acids, casein, gelatin, gluten, polyanhydrides, polyacrylic acid, alginate, chitosan, and any copolymers thereof, or any combination thereof.

Physiological functions of cells can be greatly influenced by the cell culture environment. It can be beneficial to culture the cells in a system that can best maintain the specific functions of the cells. Compared to a traditional two dimensional (2D) cell culture on the flat surface, a three-dimensional (3D) cell culture in various matrices, such as Matrigel®, has a demonstrated clear utility in maintaining cellular functions because it closely mimics the in-vivo cellular environment. Pore size and matrix stiffness have been recognized as two key factors that regulate the cellular morphology and functions in 3D cell culture system (see e.g., C. S. Ranucci, et al., Biomaterials 21(2000) 783-793; M. H. Zaman, et al., Proc Nati Acad Sci USA 103(29) 2006, 10889-94; T. Sun, et al., “Investigation of fibroblast and keratinocyte cell-scaffold interactions using a novel 3D cell culture system”, Journal of Materials Science: Materials in Medicine, 18 (2), 2007, pp. 321-328). However, precise control and modulation of the pore sizes in these culture systems can be challenging.

In embodiments, the disclosure provides compositions and methods to control and modulate the pore size and stiffness of 3D cell culture substrates to regulate cellular functions of specific cells.

In embodiments, the disclosure provides a design and method of making porous synthetic material substrates that can regulate cellular functions in 3D cell culture. In addition to providing an in vivo-like environment for cells to grow and develop their biological functionality outside of living entities, the pore size and stiffness of these porous substrates can also be controlled and modulated to regulate the cellular functions, which can serve various needs of versatile cell-based applications. For example, induction testing of certain drug agents requires relatively lower basal expression levels of target genes while inhibition testing of drug agents requires relatively higher basal expression levels of target genes. The ability to modulate gene expressions levels by tuning the pore size and stiffness properties of the substrate provides a useful platform to compare these results while having minimal disturbances on other culture conditions.

In embodiments, the disclosure provides materials having properties, such as pore size and stiffness of a porous scaffold, that can be modified to provide optimized culture performance for different cells lines; and methods for controlling pore size and stiffness of the porous scaffold that can regulate cell growth and morphology; and the scaffolds can selectively regulate the cellular function at the gene expression level.

The disclosed porous polymer substrates can provide the following features for 3D cell culture:

Pore Size Control Pore sizes can be easily controlled and modulated, which provides well controlled physical regulation of cell-cell interaction and organization.

Cell Ordering Organization and interaction of cells during cell culture influence the cell growth and functional development of the cells, therefore cellular functions can be regulated by modulating the pore sizes.

Cell Regulation Regulation of cellular functions by pore size control introduces minimum disturbance of the cellular physiological environment. Cells from the same source and can be functionally regulated by different substrates pore size properties and can provide models for comparison. For example, human hepatocyte cells cultured in the disclosed porous PDMS substrates having smaller pores have demonstrated moderate levels of CYP (Cytochrome P450) genes, such as CYP 1A2, CYP2B6, and CYP3A4, but elevated drug response induced by rifampin (USAN) or rifampicin (INN), which is well suited for induction study of cellular function by drug candidates. Human hepatocyte cells cultured in the porous PDMS having larger pores were demonstrated to have elevated level of CYP genes, which situation is ideal for inhibition study of cellular function by drug candidates.

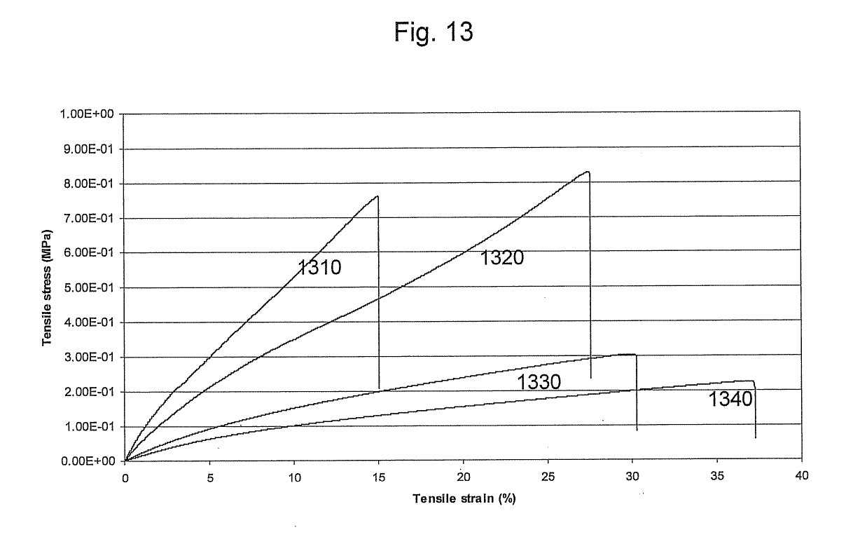

Substrate Modulation and Gene Regulation Stiffness, measured for example as Young's modulus, can be precisely controlled and modulated for the disclosed porous substrates. Modulation of the substrate's stiffness can selectively regulate cellular functions at the gene expression level. Regulation of cellular functions can be cell-specific. The easy-modulation of pore size and stiffness for the disclosed substrates provides a highly adaptable and versatile 3D cell culture platform. Young's modulus is one measure of the stiffness of a polymer material. The higher the modulus the stiffer the material. This can be experimentally determined from the slope of a stress-strain curve created during tensile tests conducted on a sample of the material. A stress-strain curve plots the material elongation (strain) as a function of force (stress) applied to the material until it fails (e.g., breaks); see FIG. 13. If the slope is steep, the sample has a high tensile modulus, which means the material resists deformation and has high stiffness. If the slope is gentle, then the material has a low tensile modulus, which means the material is easily deformed and has low stiffness. Modulus can be expressed in units of strength, such as Pa, or N/cm². The level of gene expression of the live-cells has been shown to increase with the Young's modulus stiffness of the porous polymer layer. In embodiments, the porous polymer layer can have, for example, a Young's modulus of from about 0.1 Mpa to about 15 Mpa, including intermediate values and ranges.

Cellular functions can be greatly influenced by the cell culture environment. In a well designed cell culture system, the cells can maintain their specific cellular functions. In addition, cell functions can be regulated by modulating their physical interactions with the culture environment.

In embodiments, the disclosure provides methods for regulating cellular functions in 3D cell culture by modulating the pore size and modulus (stiffness) of porous culture substrates.

In embodiments, the pore sizes of the main pore population can be larger than single cells. Therefore, cells tend to seed, migrate, proliferate, and organize inside the pores. The substrate pore size distribution can significantly influence cellular functions as a physical constraint to the cell-cell and cell-matrix interactions. The disclosed porous PDMS has a microporous structure that allows cells to grow into and within the pores and encourages cell-cell interaction. Photographic images of spheroid formation of primary human hepatocyte as a function of substrate pore size were obtained (color images not shown). The images indicate that as the porous PDMS pore size range increases from 180-to-212 microns to 300-to-355 microns, single spheroid tends to form in individual pore, and spheroid size increases with increase of the pore size; then from 500-to-600 microns, 850-to-1,000 microns, and 1,000-to-1,400 microns, instead of forming a larger spheroids in individual pores, multiple smaller spheroids are formed. In substrates of pores of size larger than 1,000 microns, less spheroids are observed and cells tend to form aggregates with loose cell-cell adhesion.

Basal gene expression experiments for genes ABCB1 (ATP-binding cassette, sub-family B (MDR/TAP), member 1), ABCC2 (ATP-binding cassette, sub-family C (CFTR/MRP), member 2), ALB (albumin), CEBPA (CCAAT/enhancer binding protein (C/EBP), alpha), GJB1(gap junction protein, beta 1), HNF4A(hepatocyte nuclear factor 4, alpha), and UGT1 (UDP glucuronosyltransferase 1), against collagen and Matrigel® controls, in human hepatocytes cultured in the disclosed porous PDMS having various pore sizes (bar chart not shown) consistently indicated that all ten gene markers are positively expressed by real-time quantitative polymerase chain reaction (RQ-PCR). These genes are a set of markers to measure how well the functions of hepatocytes are preserved in the substrates compared to industry standards, collagen surface and Matrigel®. Higher expression of all ten markers compared to the collagen surface indicates that the porous PDMS can preserve hepatocyte functions better than the collagen. More importantly, the expression level of these genes are comparable to Matrigel®, a leading commercially available substrate for preserving hepatocyte functions. In the figures, “RQ” on the y-axis represents “relative quantification” of the gene expression level of a specific gene marker divided by expression level of a house keeping gene hypoxanthine guanine phosphoribosyl transferase 1 (HPRT1).

### EXAMPLES

The following examples serve to more fully describe the manner of using the disclosure, and to further illustrate and demonstrate specific examples of best modes contemplated for carrying out various aspects of the disclosure. These examples do not limit the scope of the disclosure, but rather are presented for illustrative purposes.

### Methods For Making Cell Culture Substrates in Multi-Well Plate Format.

Multiple units each having the inventive porous PDMS can be directly formed on a glass insert of a standard holey-plate multi-well assembly as described below.

a. Direct Formation of the Porous Polymer Units on the Supporting substrates, Such as a Multi-Well Holey Plate.

Mix into a uniform mixture the selected monomer(s) or oligomer(s), optional cross-linker, and pore former particles, for example, having a single, double, or multiple particle size distribution.

Properly pack the pore-former by, for example centrifugation, shaking, pressure, or like methods.

Place a mold of desired multi-unit format, such as a multi-well holey plate, on top of the supporting substrates, such as a glass sheet of thickness less than about 500 microns.

Pour the uniform mixture of monomer or oligomer packed with pore former into the mold space.

Cure the uniform mixture in the mold at specified curing conditions.

Physically remove the cured mixture from the mold.

Remove the pore-former from the cured mixture.

Attach the supporting substrate to a holey-plate, such as with a proper adhesive or a physical retainer member.

b. Direct Formation of Porous Polymer on the Bottom of a Multi-Well Plate.

Dispense a mixture of the selected monomer or oligomer(s) and cross-linker into each well of a multi-well plate.

Dispense the pore-former into each well, and shake the plate for about 30 minutes.

Continue dispensing pore-former into each well and shake until the monomer or oligomer mixture is saturated with the pore-former, such as when a layer of pore-former remains or stays on the surface of the mixture of monomer or oligomer and pore-former after an additional 30 minutes of shaking.

Cure the polymer and pore-former mixture at the specified curing conditions.

After curing, remove the pore-former using the proper leaching condition.

c. Preform the Porous Polymer and Then Distribute it Into Each Well in a Multi-Well Plate.

Mix the selected monomer or oligomer, cross-linker, and pore-former(s) having one or two particle size distributions (i.e., mono-modal or bi-modal) in a container.

Pack the pore former and polymer precursor.

Cure the mixture to cross-link the polymer.

Leach the pore former from the cured polymer.

Cut the resulting porous polymer into the desired shape and size and place the piece on the bottom of the well plate.

Apply a fastener, such as an inert adhesive to secure the porous polymer sample to the bottom of the plate.

### Evaluation of Hepatocyte Cell Culture in a Porous Polymer (PDMS)

Attachment or viability study of hepatocyte cell line C3A in the porous polymer. Lactate Dehydrogenase (LDH), a stable cytosolic enzyme, is released upon cell lysis. The amount of the released LDH is proportional to the number of the lysed cells. A CytoTox 96 cytotoxicity assay kit (Promega) was used to evaluate the attachment of C3A cells in the porous polymer substrates by quantifying the number of viable cells. A collagen cell culture plate and a MatriGel® 3D Culture were used as control standards.

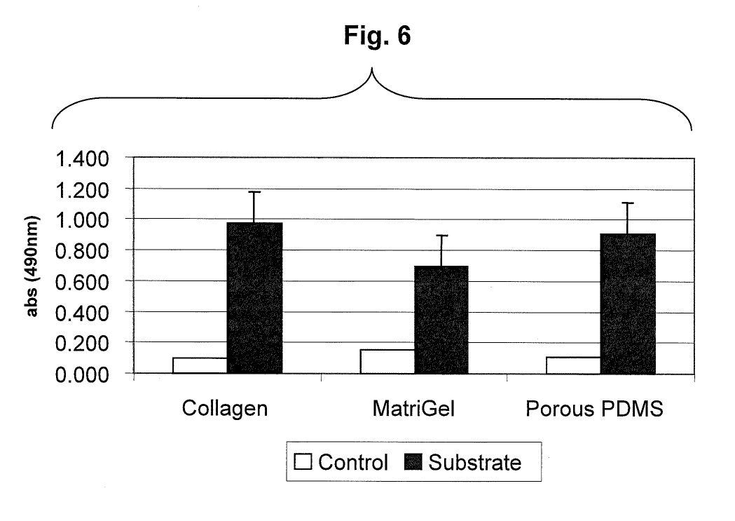

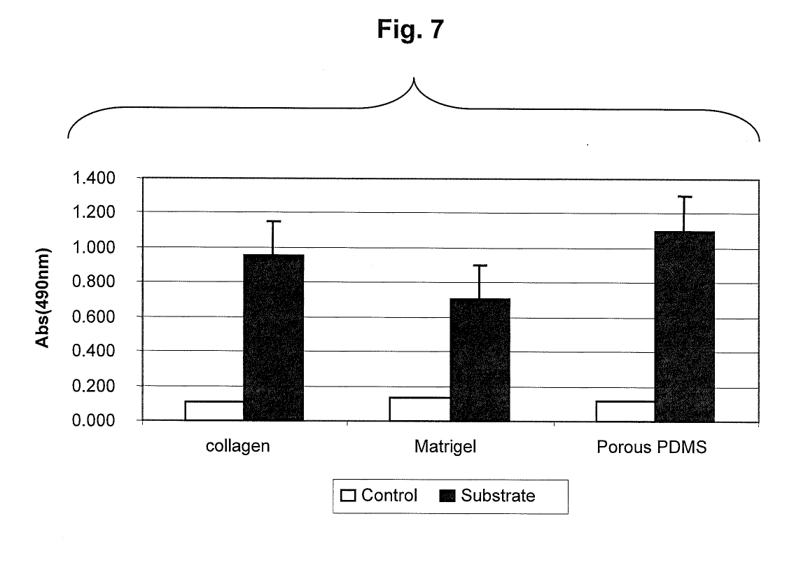

FIG. 6 shows cell attachment evaluation results with an LDH assay after 24 hours culture of hepatocyte cell line C3A on a porous PDMS polymer substrate. Similar results were observed for an LDH assay after 7 days culture and as shown in FIG. 7. Based on the LDH assay results one can readily conclude that Hepatocyte 3A4 cell lines can be cultured in the disclosed porous polymer substrates, and the attachment of the cells is comparable to the collagen standard.

### Attachment or Viability Study of Human Primary Hepatocytes

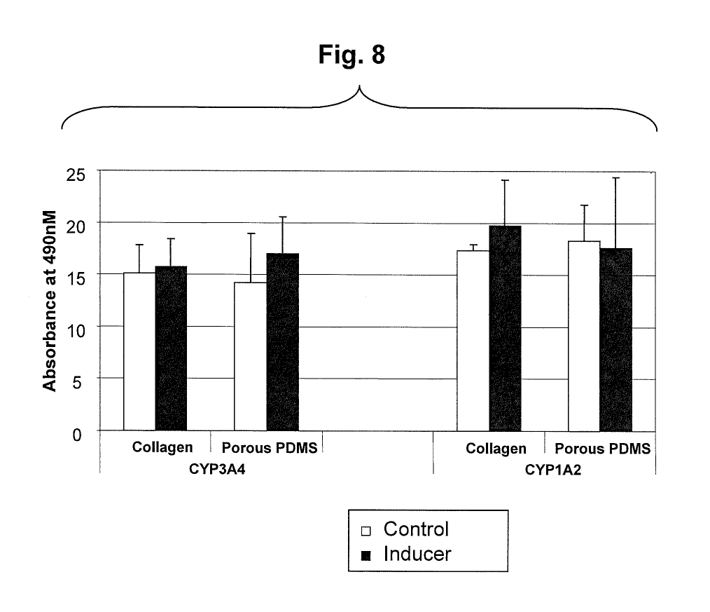

FIG. 8 shows the results of an LDH assay of human primary hepatocytes cultures in the comparative collagen and the inventive porous polymer (PDMS) with and without inducers. The inducer was rifampin at a final concentration of 10 micromolar.

### Gene Expression Analysis of Human Primary Hepatocytes Cultured in Porous PDMS Using Collagen as Normalization Standard

Maintenance of long term cultures of functionally stable primary human hepatocytes is a major goal in drug metabolism studies for drug discovery. Gene expression of various gene markers is a reliable way to evaluate the corresponding cell function(s). In the disclosed analysis, quantitative real-time PCR was used to identify the gene expression at the mRNA level. Ten gene markers listed in the Table 1 were selected based on their roles in hepatocytes-specific functions.

TABLE 1

| Gene | Gene Function |
|---|---|
| ABCB1 (900) | Transporter |
| ABCB2 (905) | Transporter |
| ALB (910) | Albumin protein |
| CEBPA (915) | transcription factor |
| CYP1A2 (920) | Metabolism-phase I |
| CYP2B6 (925) | Metabolism-phase I |
| CYP3A4 (930) | Metabolism-phase I |
| GJB1 (935) | Signal transduction |
| HNF4a (940) | Transcription factor |
| UGT1 (945) | Metabolism-phase II |

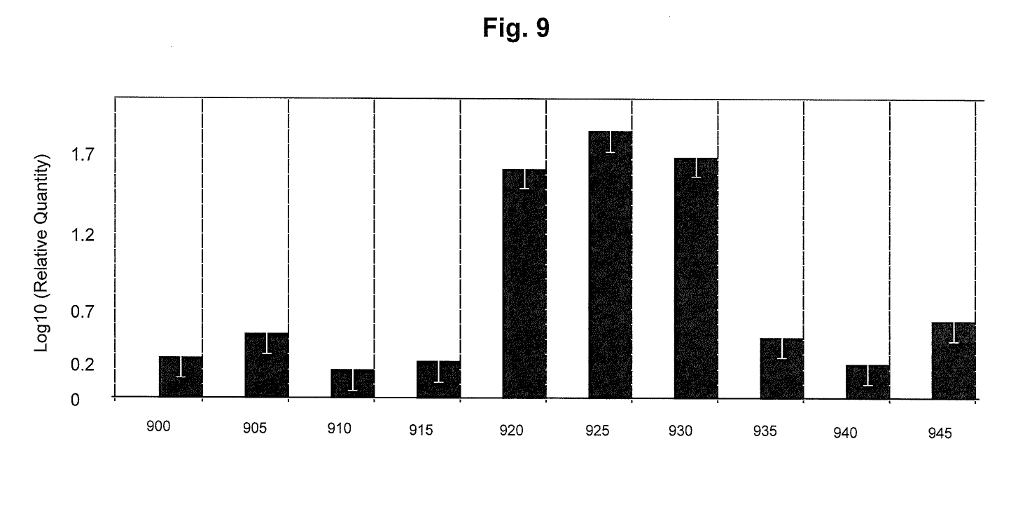

FIG. 9 shows gene expression analysis results of human primary hepatocyte in the disclosed compositions against collagen as a normalization standard. FIG. 9 results demonstrate that human primary hepatocytes cultured in the inventive porous PDMS express all 10 gene markers that were used to express hepatocytes measured as log10 (relative quantity) of expression: ABCB1 (900), ABCB2 (905), ALB (910), CEBPA (915), 1A2(920), 2B6 (925), 34A (930), GJB1 (935), HNF4a (940), and UGT1 (945).

### Example 1

Culture Media Preparation Polydimethylsiloxane (PDMS) was used as a base material to demonstrate the aspects of making and using the disclosed culture media. A Sylgard-182 PDMS elastomeric kit from Dow-Corning was used to prepare

PDMS related materials. A 9:1 by weight Sylgard-182A/Sylgard-182B mixture was mixed with sugar crystals in a 1:3 or slightly higher volume ratio of the desired size distribution. Sugar crystals having the desired size distribution were obtained by sieving granulated commercial sugar (sucrose) with sieves of various sizes, and then placed in a centrifuge for one hour at 2,400 rpms to closely pack the sugar crystals in the PDMS pre-polymer, and then degassed in vacuum before cured for about 3 hours at 75° C. The sugar crystals were leached from the cured PDMS polymer matrix using deionized water in a heated ultrasonic bath at a temperature of about 40 to about 70° C. for about 8 hours. The leached matrix was washed with deionized water then dried under vacuum in a sterile environment for about 24 hours. The resulting porous PDMS material was examined microscopically and found to have an interconnected pore structure and high optical transparency. The porous PDMS material was easily cut, for example, with scissors, a laser, a punch, and like implements and methods, into desired size(s) and thickness(es) for cell culture purposes.

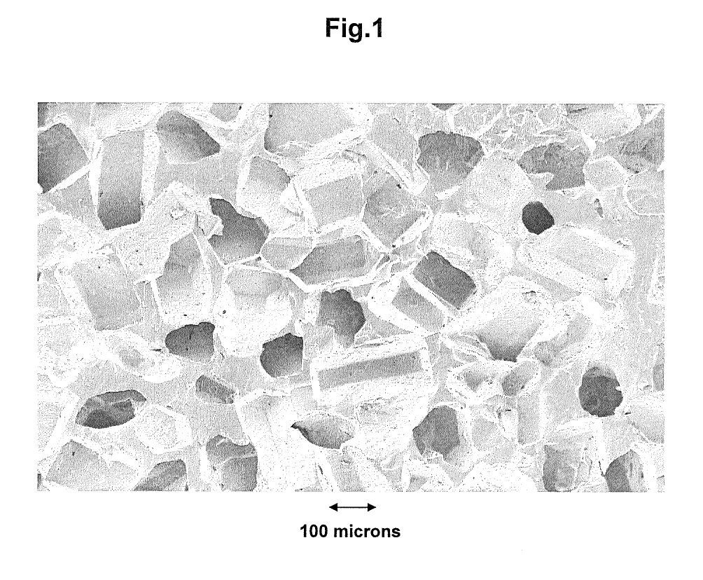

Optical Image Analysis Referring to the Figures, FIG. 1 shows an exemplary porous PDMS article having an interconnected pore structure as imaged by a confocal microscope in reflection mode.

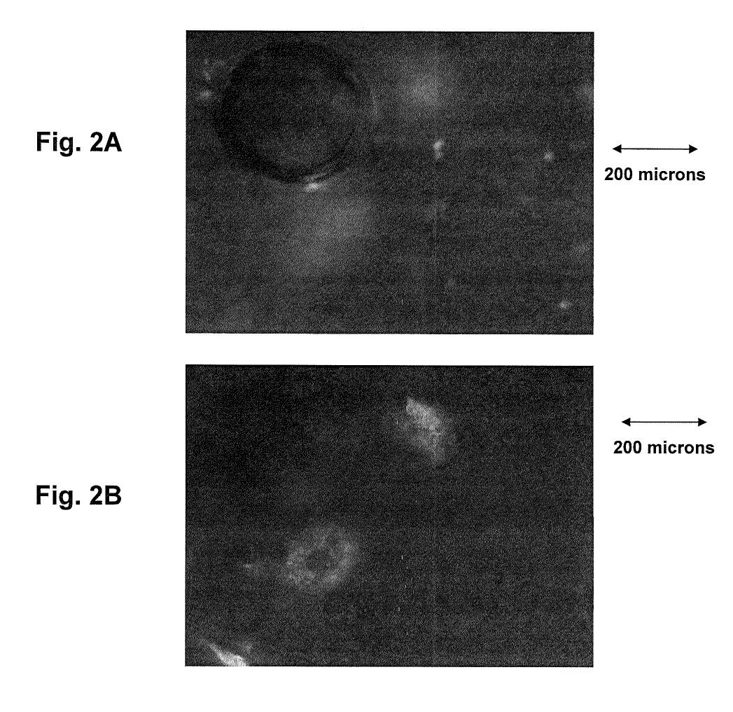

FIG. 2A highlights hepatocyte spheroid formation in the inventive porous PDMS substrate. The magnified (20×) surface image shows a pore opening of about 150 to about 180 microns in diameter (in-focus; top-left) on the outer surface of the porous PDMS and some scattered single hepatocyte cells. Cultured HepG2 spheroids foamed beneath the surface and within the interstices of the article (out-of-focus).

FIG. 2B shows HepG2 spheroids that formed during culture in the porous PDMS cell culture article of FIG. 2A having shifted the magnified (20×) focus from the article surface to the inside of the article to highlight the in-focus HepG2 spheroids.

### Comparative Example 1

Polyvinyl alcohol (PVA) substrates Example 1 was repeated with the exception that the porous PVA substrate was obtained commercially. Porous PVA substrates, such as high porosity PVA sponge, are commercially available from, for example, Ceibatech (www.ceibatech.com) and as disclosed in U.S. Pat. No. 5,554,659. A two-photon fluorescence microscope image of the porous PVA substrate provided only about 90 microns of penetration.

### Example 2

Hep G2 Cell Culture The viable hepatocytes C3A cell line (having less than or equal to 10× passages) cells were plated in DMEM medium with 10% FBS and 1% penicillin on porous PDMS surface along with controls, commercially available collagen and Matrigel® pre-coated surfaces. The seeding density was about 100K cells per well. The cultures were incubated for 24 hrs at 37° C. for attachment. The medium was changed at 24 hours, and on the 3^{rd} and 7^{th} days. The porous PDMS coated substrate of Example 1 was shown to be useful in hepatocytes spheroids culture in 3D.

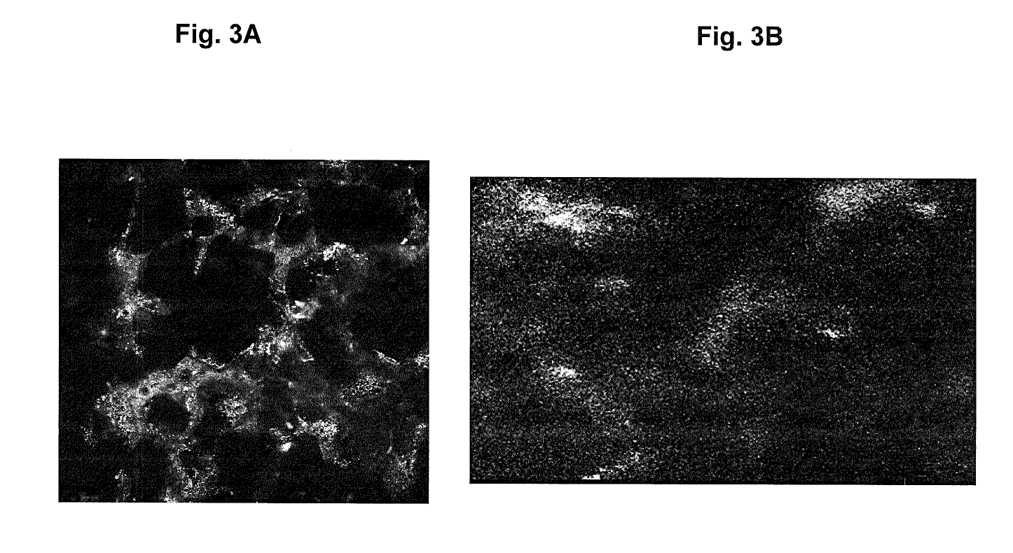

FIG. 3A shows an exemplary sample of a porous PDMS article having interconnected pore structure imaged by a confocal microscope in reflection mode and prepared according to Example 1.

FIG. 3B shows a two-photon fluorescence image of the sample of FIG. 3A doped with Qdot at depth of 530 microns. This sample and image demonstrate the enhanced imaging penetration depth offered by the porous PDMS substrates of the disclosure. A two-photon fluorescence microscope was used to image a porous PDMS sample that was doped with 5 microg/mL Qdot® fluorescent nanocrystals (available for Invitrogen; see www.invitrogen.com) having 460 nm emission, good optical signal was detected at the depth of 530 microns within the substrate as shown in FIG. 3B.

FIG. 3A shows a pore opening (in-focus) on the surface of a porous PDMS substrate and hepatocytes spheroids formed underneath the surface, i.e., within the substrate (out-of-focus). In FIG. 3B the focus was shifted from the substrate surface down to the inside of the substrate to focus on the spheroids. The FIG. 3A and 3B images demonstrated that the HepG2 cells were able to seed and migrate within the interconnected porous structure of the PDMS substrates, and form into cell spheroids which reproduce the in-vivo behavior of HepG2.

### Example 3

Primary Hepatocytes Culture Cryopreserved primary hepatocytes purchased from XenoTech, LLC, were thawed and purified using a Percoll isolation kit (XenoTech, LLC.). The viable cells were plated in 10% FBS containing MFE medium (Corning Inc.) on a porous PDMS surface along with controls, collagen and Matrigel® pre-coated surfaces. The seeding density was about 400K cells per well. The cultures were incubated for 18 hrs at 37° C. for attachment. The medium was changed to serum-free MFE medium for remainder of the culture period. Rifampin induction for CYP3A4 metabolic activity was performed for three consecutive days with daily medium change. The CYP3A4 metabolic activity in each surface culture was evaluated by either the HPLC method after three hours of incubation with testosterone (200 microM) or measured using a Promega P450™ kit. The cell number in the cultures was estimated using a Promega CytoTox 96 Non-Radioactive cytotocity LDH assay kit.

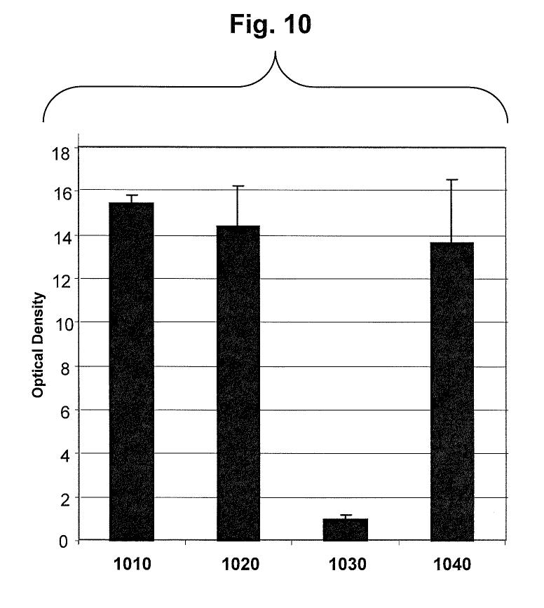

FIG. 10 compares the number of viable human hepatocyte cells as measured by optical density after 7 days of culture with various culture surface: Collagen (2D)(1010), Matrigel®(3D) (1020), Monolayer PDMS (2D) (1030), and the porous PDMS (3D) (1040) of the disclosure. The porous PDMS (3D) (1040) of the disclosure was comparable to the collagen (2D)(1010) and Matrigel®(3D) (1020), and exceptional compared to the monolayer PDMS (2D) (1030).

### Example 4

Porous PDMS substrates of pore sizes ranging from 180 microns to 1,400 microns were fabricated in accordance with the disclosure to demonstrate that pore size can regulate hepatocyte cellular functions in cellular organization and RNA expression. A mixture of PDMS pre-polymer and curing agent in a mixing ratio of 10 to 1 was closely packed with sugar crystals having well defined size distribution ranges, such as from about 75 microns to 1,400 microns, and then cured at 100 degrees C. for about one hour. Lower curing temperatures can be used if curing time is extended. The sugar in the cured polymer was dissolved with water and washed out in an ultrasonic bath to form porous PDMS substrates for 3D cell culture.

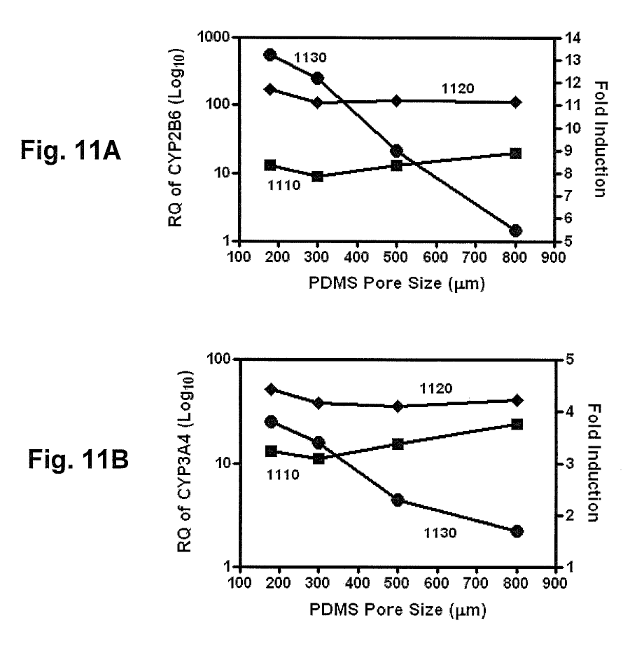

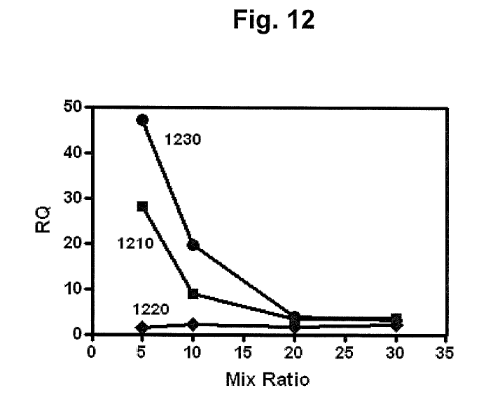

Primary human hepatocytes were seeded and cultured in these pore size controlled porous PDMS substrates. A time course study of the hepatocyte morphology revealed that the pore size of the porous substrates regulated the formation of the hepatocyte spheroids in the culture. In the porous substrates having smaller pore sizes, hepatocytes tended to form one spheroid inside each individual pore and the spheroid size increased with the size of pores. In pores larger than about 355 microns, multiple smaller spheroids tended to form in each pore instead of forming a large spheroid. In addition to regulating the formation of spheroids in the porous substrates, the results of a real time polymerase chain reaction (PCR) (FIG. 11) revealed that the gene expression level of gene CYP2B6 and gene CYP3A4 echoed the trend of the spheroid formation, i.e., the gene expression levels of both genes decreased when substrate pore size was increased from 180 microns to 355 microns. Gene expression levels increased with the pore size when pore size was larger than 355 microns. Furthermore, the induction fold of the gene expression by rifampin (FIG. 12) decreased with the pore size from 180 microns to 1,400 microns. FIGS. 11A and 11B show gene expression levels of human hepatocytes induced by rifampin in porous PDMS substrates having various pore sizes. FIG. 11A shows gene expression of gene CYP2B6 and FIG. 11B shows gene expression of gene CYP3A4, where the curves (1110), (1120), and (1130) represent basal, induction, and fold induction, respectively.

From at least the foregoing results, it was concluded that: 1) the pore size of the porous 3D cell culture substrates can regulate the expression level of selective genes while maintaining the expression level of others; 2) the substrate pore size can also regulate the cellular response to a drug reflecting differences in induction fold (i.e., the gene expression level with the inducer (drug) divided by basal expression level (without inducer); 3) porous substrates having pore size smaller than about 355 microns promotes a high induction response to a drug, which can be used in induction applications with various chemicals on the cells; and 4) porous substrates having pore size larger than about 355 microns promote elevated gene expression of selective genes, which can be effectively used in inhibition assays of cell-drug interaction.

### Example 5

Regulating the cellular functions by modulating the stiffness (modulus) of the porous substrates The shear modulus of PDMS varies with preparation conditions, but is typically in the range of 100 kPa to 3 Mpa. The stiffness (modulus) of PDMS decreases with the mixing ratio of pre-polymer and curing agent. Mixtures of pre-polymer and curing agent in a mixing ratio by weight percent of 5, 10, 20, and 30 were used to make porous PDMS substrates following the procedure in Example 4. Human primary hepatocytes were seeded and cultured for seven days. FIG. 12 shows gene expression of human hepatocytes cultured in porous PDMS having pore sizes of from about 180 to about 212 microns and having various stiffness based on the mixing ratio of pre-polymer and curing agent: CYP1A2 (1220), CYP2B6 (1210), and CYP3A4 (1230). The stiffness of PDMS decreases with the increase of the mixing ratio of PDMS pre-polymer base to curing agent. The substrate stiffness, as characterized by mixing ratios, that were evaluated and shown in FIG. 12 were, for example, 5:1, 10:1, 20:1, and 30:1. The results of real time PCR of the cells in FIG. 12 demonstrated that gene expression levels of CYP2B and CYP34A decreased with an increase in the mixing ratio of the PDMS pre-polymer to curing agent, i.e., gene expression levels increased with the stiffness of the porous substrates.

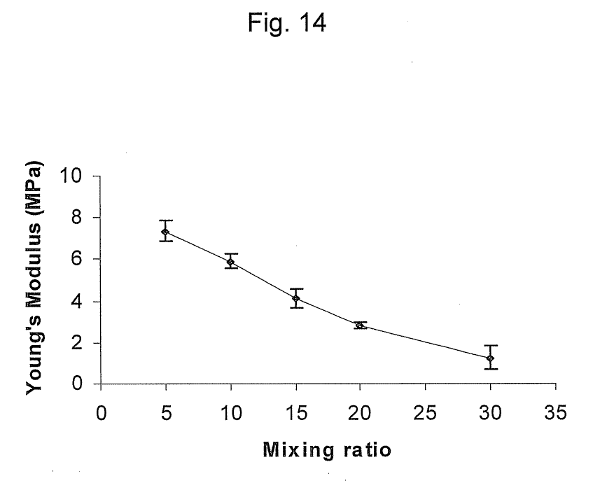

FIG. 13 a shows a relationship of the measured modulus (stress versus strain) for porous PDMS substrates prepared having various mixing ratios of the prepolymer (monomer or oligomer) to curing agent: 5:1 (1310); 10:1 (1320), 20:1 (1330), and 30:1 (1340). FIG. 14 shows the relationship of the measured modulus of curves for the porous PDMS substrates of FIG. 13 as a function of the mixing ratio.

The disclosure has been described with reference to various specific embodiments and techniques. However, it should be understood that many variations and modifications are possible while remaining within the spirit and scope of the disclosure.

What is claimed is:

1. A method for making a three-dimensional porous cell culture article, the method comprising:
polymerizing a mixture comprising at least one monomer, an oligomer, or a mixture thereof, and at least one particulate pore-former to form a continuous polymer matrix having a close-packed particulate phase; and
treating the resulting solid matrix to remove the close-packed particulate phase from the matrix.

2. The method of claim 1 wherein the at least one particulate pore-former comprises at least one of:
a first particle mixture having a particle diameter of from about 75 micrometers to about 1,000 micrometers;
a second particle mixture having a particle diameter of from about 0.1 micrometers to about 75 micrometers;
or a combination thereof.

3. The method of claim 2 wherein the first particle mixture and the second particle mixture are independently selected from mono-modal particles, bimodal particles, mono-disperse particles, bi-disperse particles, poly-disperse particles, or a combination thereof.

4. The method of claim 1 wherein polymerizing the mixture is accomplished on a substrate, and the resulting three-dimensional porous cell culture article being substantially optically transparent.

5. The method of claim 1 wherein polymerizing the at least one monomer or oligomer comprises forming a continuous polymer phase from at least one monomer or oligomer selected from the group consisting of a siloxane, a vinyl substituted trialkoxy silane, an alpha-olefin, a vinyl ester, an acrylate, an acrylamide, an unsaturated ketone, a monovinylidene aromatic hydrocarbons, or a combination thereof.

6. The method of claim 1 wherein the at least one pore-former is selected from particles of a sugar, a polysaccharide, a polyalkylene glycol, a polyvinylalcohol, ice, a wax, a substance having a melting point lower than that of the polymer formed, a water soluble polymer, a water-insoluble polymer, or a copolymer of a water-insoluble monomer or oligomer and a water-soluble monomer or oligomer, a microcapsule having a shell and core, a microballoon having a shell and hollow core, or combinations thereof.

7. The method of claim 1 wherein treating the resulting solid matrix to remove the particulate phase from the matrix comprises at least one of:
contacting with a substance to dissolve the particulate phase, the substance comprises at least one of: an aqueous liquid, an organic liquid, a supercritical fluid, a low melting solid, a gas, or a combination thereof;
heating the matrix to liquefy the particulate phase;
sonicating;
or a combination thereof.

8. The method of claim 7 wherein the resulting polymer phase less the particulate phase has a refractive index of from about 1.2 to about 1.45.

9. The method of claim 1 further comprising selecting a pore-former packing density based on a particle size ensemble having a void volume filled with a polymerizable monomer, oligomer, or mixture thereof, that becomes the continuous polymer matrix and the volume-fraction occupied by the particulate pore-former that becomes the void-volume in the resulting cell culture article.

10. The method of claim 1 wherein the mixture comprising at least one monomer or oligomer for polymerization and at least one particulate pore-former being prepared by at least one of high speed liquid mixing, blending, centrifuging, or a combination thereof.

11. A three-dimensional cell culture article comprising:
a polymer mass having an interconnected porous network, the interconnected porous network comprising pores comprised of a monomodal distribution of pore sizes and their corresponding interstices, a bimodal distribution of pore sizes comprised of larger pores and smaller pores and their corresponding interstices, or a combination thereof, and the article being substantially optically clear.

12. The article of claim 11 wherein the polymer mass comprises at least one of: a bead; a reconstitutable powder; a coating formulation; a thin film of thickness of from about 20 micrometers to about 500 micrometers; a porous monolith; or a combination thereof.

13. A three-dimensional cell culture article prepared by the process of claim 1, comprising:
a substrate; and
a polymer layer having an interconnected porous network supported on the substrate,
the porous polymer layer comprising a continuous polymer matrix having a continuous or semi-continuous void phase.

14. The article of claim 13 wherein the porous polymer has a surface area of from about 1 to about 20 m²/g.

15. The article of claim 13 wherein the porous polymer has a porosity of from about 50% to about 95% as measured by mercury porosimetry, has a refractive index in air of from about 1.28 to about 1.45, has a density of from about 0.5 to about 1.5 kg/m³, and has a molecular weight of from about 500 to about 500,000 Daltons.

16. The article of claim 13 wherein the porous polymer has an optical density from about 0 to about 1, and an optical penetration depth of from about 10 to about 1,000 microns.

17. The article of claim 13 further comprising at least one additive selected from the group consisting of a nutrient, an antibiotic, a growth stimulator, a growth inhibitor, a surface modifier, a surface compatibilizer, or a combination thereof.

18. A cell culture method comprising: contacting the cell culture article of claim 13, with culture media, and then live-cells.

19. The method of claim 18 wherein the resulting cell culture provides a retention rate of from about 90 to about 100 percent.

20. The method of claim 18 wherein the level of gene expression of the live-cells increase with the Young's modulus stiffness of the porous polymer layer, the porous polymer layer having a Young's modulus of from about 0.1 Mpa to about 15 Mpa.
